(19) United States
(12) Patent Application Publication
(10) Pub. No.: US 2009/0208991 A1
(43) Pub. Date: Aug. 20, 2009

(54) Prediction of bone marrow toxicity

(75) Inventors: Hans Marcus Ludwig Bitter, San Francisco, CA (US); David Michael Goldstein, San Jose, CA (US); Kyle Louis Kolaja, San Mateo, CA (US); Henry Lin, Cupertino, CA (US); Andrew James Olaharski, Sunnyvale, CA (US); Hirdesh Uppal, Los Altos, CA (US)

Correspondence Address: Grant D. Green, Patent Law Department;M/S A2-250, Roche Palo Alto LLC, 3431 Hillview Avenue, Palo Alto, CA, 94304, US

(73) Assignee: Roche Palo Alto LLC

(21) Appl. No.: 12/378,437

(22) Filed: Feb. 12, 2009

Related U.S. Application Data
(60) Provisional application No. 61/028,742, filed on Feb. 14, 2008.

(51) Int. Cl. C12Q 1/48

(52) U.S. Cl. 435/15

(57) ABSTRACT

The likelihood that a compound will exhibit bone marrow toxicity in an in vivo assay predicted by the ability of the compound to inhibit at least eight kinases from a selected group.

This application claims priority from copending application U.S. Ser. No. 61/028,742, filed on Feb. 14, 2008, incorporated herein by reference in full.

### FIELD OF THE INVENTION

This invention relates generally to the field of toxicology. More particularly, the invention relates to methods for predicting bone marrow toxicity, and methods for screening compounds for potential bone marrow toxicity.

### BACKGROUND OF THE INVENTION

Bone marrow ablation is often observed during in vivo toxicity studies for potent cytotoxic pharmaceutical compounds because progenitor bone marrow cells are highly proliferative and susceptible to cell cycle arrest, DNA damage, and apoptosis. Bone marrow toxicity is a major concern, particularly for drugs developed for indications other than oncology, as it can lead to neutropenia, anemia, and general immunosuppression. Thus, compounds that ablate bone marrow during in vivo toxicity studies are often dropped from further development, resulting in program delays and substantial financial expenditures.

Performing in vivo toxicological studies to determine bone marrow ablation is laborious, time consuming, expensive, and typically requires large quantities of compound. In vitro assays measuring specific progenitor stem-cell population toxicity and/or colony formation can be used as surrogates for in vivo toxicity studies, but these methods require further validation to address whether they can recapitulate the complexities and nuances observed with an in vivo study.

Kinases are enzymes responsible for phosphorylating substrates and disseminating inter- and intracellular signals. They fulfill integral roles in progenitor stem-cell differentiation as well as the initiation, propagation, and termination of mitosis in hematopoietic progenitor stem cells. Kinases are often the target of pharmaceutical research because many signaling cascades have known roles in a variety of diseases. Small molecule kinase inhibitors (SMKIs) often competitively bind to the kinase ATP binding pocket, blocking the ability of the enzyme to phosphorylate substrates. SMKIs often inhibit many kinases in addition to the desired target, due to the highly conserved nature of the ATP binding pocket within the kinome, thus toxicities associated with off-target kinase inhibition is a concern for this class of compounds. In particular, bone marrow toxicity or ablation, observed in the clinic or in in vivo toxicity studies, is a common toxicological liability for SMKIs because the kinases responsible for cellular differentiation or proliferation can be inhibited.

### SUMMARY OF THE INVENTION

We have now invented a method for predicting which compounds will demonstrate positive (i.e., bone marrow toxicity) results in in vivo bone marrow toxicity studies, using a method that is faster, uses smaller quantities of reagents, is easily automated, and is much cheaper.

One aspect of the invention is a method for predicting the in vivo bone marrow toxicity of a compound, said method comprising providing a test compound; and determining the ability of said compound to inhibit the kinase activity of a set of predictive kinases, wherein each predictive kinase is selected from the group consisting of ANKK1, AURKC, CLK4, IRAK3, JAK1, MARK2, MUSK, MYLK2, RIPK1, ROCK2, STK17A, STK17B, SGK110, TRKA, TRKC, ULK1, ULK2, ZAP70, and TYK2; wherein inhibition of kinase activity of at least eight predictive kinases by 85% or greater constitutes a prediction that said compound would exhibit bone marrow toxicity in vivo.

Another aspect of the invention is a method for developing drugs, comprising: providing a plurality of compounds; determining the ability of each compound to inhibit the kinase activity of a set of predictive kinases, wherein each predictive kinase is selected from the group consisting of ANKK1, AURKC, CLK4, IRAK3, JAK1, MARK2, MUSK, MYLK2, RIPK1, ROCK2, STK17A, STK17B, SGK110, TRKA, TRKC, ULK1, ULK2, ZAP70, and TYK2; and rejecting each compound that demonstrates inhibition of kinase activity of a threshold number of predictive kinases by about 85% or greater at about 10 μM.

Another aspect of the invention is a substrate for testing compounds for potential bone marrow toxicity, comprising a surface having bound thereto a set of predictive kinases selected from the group consisting of ANKK1, AURKC, CLK4, IRAK3, JAK1, MARK2, MUSK, MYLK2, RIPK1, ROCK2, STK17A, STK17B, SGK110, TRKA, TRKC, ULK1, ULK2, ZAP70, and TYK2.

### DETAILED DESCRIPTION OF THE INVENTION

All publications cited in this disclosure are incorporated herein by reference in their entirety.

### Definitions

Unless otherwise stated, the following terms used in this Application, including the specification and claims, have the definitions given below. The singular forms “a”, “an,” and “the” include plural referents unless the context clearly dictates otherwise.

The term “bone marrow toxicity” as used herein refers to hypocellularity of the hematopoietic cell system, including B cells, T cells, NK cells, neutrophils, eosinophils, basophils, dendritic cells, mast cells, megakaryocytes, platelets, erythrocytes or any of their progenitors in a bird or mammal, caused by the administration of or contact with a chemical or biological agent. In most cases, the bird or mammal is a mouse, rat, beagle dog, or non-human primate used for pre-clinical safety studies, but may be a human. A “likelihood of bone marrow toxicity” means specifically that the compound in question is predicted to demonstrate bone marrow toxicity, or lack thereof, in an in vivo bone marrow test with at least 75% confidence.

The term “test compound” refers to a substance which is to be tested for bone marrow toxicity. The test compound can be a candidate drug or lead compound, a chemical intermediate, environmental pollutant, a mixture of compounds, and the like.

The term “kinase” refers to an enzyme capable of attaching and/or removing a phosphate group from a protein or molecule. “Inhibition of kinase activity” refers to the ability of a compound to reduce or interfere with such phosphatase activity. As binding affinity of a small molecule for a given kinase correlates well with the ability of said molecule to inhibit the kinase activity, binding affinity is considered synonymous with kinase activity herein, and high binding affinity is considered equivalent to high kinase inhibitory activity.

The terms “identified kinase” and “predictive kinase” refers to the set: ANKK1, AURKC, CLK4, IRAK3, JAK1, MARK2, MUSK, MYLK2, RIPK1, ROCK2, STK17A, STK17B, SGK110, TRKA, TRKC, ULK1, ULK2, ZAP70, TYK2. These kinases are further identified by the following accession numbers: NP_{—}848605.1 (ANKK1); AAC77369.1 (AURKC); NP_{—}065717.1 (CLK4); NP_{—}009130.1 (IRAK3); NP_{—}002218.2 (JAK1); NP_{—}059672.2 (MARK2); NP_{—}005583.1 (MUSK); NP_{—}149109.1 (MYLK2); NP_{—}003795.2 (RIPK1); NP_{—}004751.2 (STK17A); NP_{—}004217.1 (STK17B); P0C264 (SGK110); NP_{—}001012331.1 (TRKA); AAA75374.1 (TRKC); NP_{—}00.3556.1 (ULK1); NP_{—}055498.2 (ULK2); NP_{—}997402.1 (ZAP70); CAA38449.1 (TYK2).

### General Method

The invention provides a method for quickly determining the likelihood that a given compound will exhibit bone marrow toxicity in an in vivo toxicity assay by examining the interaction between the compound and a number of kinases (kinase binding and/or inhibition). As kinase inhibition and/or binding can be determined quickly, and by using automated methods, the method of the invention enables high-throughput screening of compounds for bone marrow toxicity (or lack thereof).

In practice, binding and inhibition can be determined using methods known in the art. See, for example, M. A. Fabian et al., Nature Biotechnol (2005) 23:329-36, incorporated herein by reference in full. In general, the binding affinity of a compound for a given kinase correlates well with the ability of the compound to inhibit the activity of that kinase, so that binding affinity is a reliable substitute for inhibitory activity. Binding affinity may be determined by a variety of methods known in the art; for example by competitive assay using an immobilized kinase (or an immobilized test compound, or an immobilized competing ligand, any of which may be labeled). Compounds and kinases can be immobilized by standard methods, for example by biotinylation and capture on a streptavidin-coated substrate.

Thus, one can prepare a test substrate having, for example, a plurality of immobilized kinases, preferably comprising the nineteen identified herein: ANKK1, AURKC, CLK4, IRAK3, JAK1, MARK2, MUSK, MYLK2, RIPK1, ROCK2, STK17A, STK17B, SGK110, TRKA, TRKC, ULK1, ULK2, ZAP70, TYK2.

The kinases can be immobilized directly (i.e., by adsorption, covalent bond, or biotin-avidin binding or the like) to the surface, or indirectly (for example by binding to a ligand that is tethered to the surface by adsorption, covalent bond, biotin-avidin or other linkage). The kinases are then contacted with the test compound(s), and the affinity (or enzyme inhibition) determined, for example by measuring the binding of labeled compound or loss of labeled competitor.

The kinase affinity of each compound is measured against the kinases comprising the model. A compound with high total activity (for example, demonstrating high affinity for eight or more of the nineteen kinases) has a high likelihood of bone marrow toxicity: this compound is predicted to test positive for bone marrow toxicity in an in vivo test system. A compound having high activity against sixteen or more of the identified kinases is very likely to demonstrate bone marrow toxicity. A compound having low total activity (for example, showing only low affinity for the identified kinases, or showing high affinity to only 1-4 identified kinases) is predicted to test negative in the toxicity assay. “High affinity” as used herein refers to inhibition of the kinase activity by at least about 85% at about 10 μM.

Candidate drugs that test positive in the assay of the invention (i.e., that are predicted to demonstrate bone marrow toxicity in the in vivo assays) are generally identified as “bone marrow ablating” or “potentially bone marrow ablating”, and rejected or otherwise dropped from further development. In the case of high-throughput screening applications, such compounds can be flagged as potentially bone marrow ablating (for example, by the software managing the system in the case of an automated high-throughput system), thus enabling earlier decision making.

Thus, one can use the method of the invention to prioritize and select candidate compounds for pharmaceutical development based in part on the potential of the compound for bone marrow toxicity. For example, if one has prepared a plurality of compounds (e.g., 50 or more), having similar activity against a selected target, and desires to prioritize or select a subset of said compounds for further development, one can test the entire group of compounds in the method of the invention and discard or reject all those compounds that exhibit positive signs of bone marrow toxicity. This reduces the cost of pharmaceutical development, and the amount invested in any compound selected for development by identifying an important source of toxicity early on. Because the method of the invention is fast and easily automated, it enables the bulk screening of compounds that would otherwise not be possible or practical.

Environmental pollutants and the like can also be identified using the method of the invention, in which case such compounds are typically identified for further study into their toxic properties. In this application of the method of the invention, one can fractionate an environmental sample (for example, soil, water, or air, suspected of contamination) by known methods (for example chromatography), and subject said fractions to the method of the invention. Fractions that display signs of bone marrow toxicity can then be further fractionated, and (using the method of the invention), the responsible toxic agents identified. Alternatively, one can perform the method of the invention using pure or purified compounds that are suspected of being environmental pollutants to determine their potential for bone marrow toxicity. Because the method of the invention is fast and easily automated, it enables the bulk screening of samples that would otherwise not be possible or practical.

The following additional kinases can also be tested: high affinity of a compound for one or more of these additional kinases (in addition to a majority of the nineteen identified kinases) correlates with a higher likelihood of bone marrow toxicity. The additional kinases are: AMPKA1 (BAA36547.1), CDK7 (NP_{—}001790.1), IKKE (NP_{—}054721.1), MLK2 (NP_{—}002437.2), MLK3 (NP_{—}002410.1), MERTK (AAB60430.1), MLCK (NP_{—}872299.1), PAK4 (NP_{—}001014833.1), SLK (NP_{—}055535.2), MST3 (NP_{—}003567.2), STK33 (NP_{—}112168.1), SYK (NP_{—}003168.2), TRKB (NP_{—}006171.2), TSSK1 (NP_{—}114417.1), JAK2 (NP_{—}004963.1)

### EXAMPLE

To identify the set of kinases that would indicate a likelihood that a test compound would demonstrate bone marrow toxicity, the following analysis was carried out. First, 65 suitable small molecule kinase inhibitors (“SMKIs”) were selected to form a training set. Second, for each compound in the training set, an in vivo test result and single point inhibition profiles against 322 kinases were acquired. A statistical analysis was then performed to (1) build a model using said single point kinase inhibition profiles to predict said bone marrow toxicity result and (2) identify the kinases correlated with bone marrow toxicity results.

Inhibition profiles against 322 kinases and in vivo assay results were acquired for each compound in the training set (N=65). Two different readouts were obtained for the assay results: negative (N=40) and positive (N=25). Pre-processing was first performed across the set of all inhibition profiles to remove uninformative or biased kinases. Kinases with no variance across the set of 65 compounds were removed, as they were not informative.

Feature selection (FS) and pattern recognition (PR) were performed in order to build the model. For all analyses, cross validation was used to assess the model performance over several trials. Each trial randomly split the initial data into a training set and a test set; the training set was used to build the temporary model, and the test set was used to predict results and then verify performance. Feature selection methods were used to determine which kinases, or “features”, were likely to correlate most with bone marrow toxicity result. In each trial, the inhibition values against the features chosen were used as input for a pattern.

A combination of a Q-value/Wilcox T-test hybrid algorithm for FS1 (Storey J D., “A direct approach to false discovery rates” (2002, J Royal Stat. Soc. B, 64: 479-498); Storey J D et al., “Statistical significance for genome-wide experiments” (2003, Proc Natl Acad Sci USA, 100: 9440-45); Storey J D., “The positive false discovery rate: A Bayesian interpretation and the q-value” (2003, Ann. Stat, 31: 2013-35); Storey J D et al., “Strong control, conservative point estimation, and simultaneous conservative consistency of false discovery rates: A unified approach” (2004, J Royal Stat. Soc. B, 66: 187-205)) and Support Vector Machines for PR (T. Hastie et al., “The Elements of Statistical Learning” (2001, Springer-Verlag); R. O. Duda et al., “Pattern Classification, 2^{nd} Ed.” (2000, Wiley-Interscience); and “Feature Extraction—Foundations and Applications” (2006, Springer-Verlag, I. Guyon et al. Eds.)).

The chosen combination of methods was used to optimize the model's performance by varying the number of kinases used as input for PR. The mean error rate was lowest when nineteen kinases were chosen.

The accuracy of the model using this combination of feature selection and pattern recognition methods, number of features, and optimal tuning parameters was then assessed by performing 10 five-fold cross-validations. Importantly, the feature selection and pattern recognition were performed within each cross-validation fold. The resulting model had an accuracy of 85%±5%: that is, the model on average correctly predicted bone marrow toxicity results 85% of the time.

The 10 five-fold cross-validations were also used to determine the kinases correlated with bone marrow toxicity result. The selection of kinases was based on the number of times a kinase was chosen as significant amongst the 50 trials (10 five-fold cross-validations) and the fact that reasonable error rates were obtained between 15-25 features. The top nineteen frequently chosen kinases were selected to be included in the final model. Over multiple runs of testing, the kinase inhibition profiles against these nineteen kinases were found to be significant in predicting actual bone marrow toxicity.

For each SMKI, the model consists of single point kinase inhibition profiles against the following nineteen kinases: ANKK1, AURKC, CLK4, IRAK3, JAK1, MARK2, MUSK, MYLK2, RIPK1, ROCK2, STK17A, STK17B, SGK110, TRKA, TRKC, ULK1, ULK2, ZAP70, TYK2. Additionally, an in vivo bone marrow toxicity assay result at the concentration in which the kinase screen was performed is included.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

All patents and publications identified herein are incorporated herein by reference in their entirety.

What is claimed:

1. A method for predicting the in vivo bone marrow toxicity of a compound, said method comprising:
a) providing a test compound;
b) determining the ability of said compound to inhibit the kinase activity of a set of predictive kinases, wherein each predictive kinase is selected from the group consisting of ANKK1, AURKC, CLK4, IRAK3, JAK1, MARK2, MUSK, MYLK2, RIPK1, ROCK2, STK17A, STK17B, SGK110, TRKA, TRKC, ULK1, ULK2, ZAP70, and TYK2;
wherein inhibition of kinase activity of at least eight predictive kinases by 85% or greater constitutes a prediction that said compound would exhibit bone marrow toxicity in vivo.

2. The method of claim 1, wherein said set of predictive kinases comprises MUSK.

3. The method of claim 2, wherein said set of predictive kinases further comprises TYK2 and IRAK3.

4. The method of claim 3, wherein said set of predictive kinases further comprises SgK110 and TRKC.

5. The method of claim 4, wherein said set of predictive kinases further comprises ZAP70 and ROCK2.

6. The method of claim 5, wherein said set of predictive kinases further comprises MYLK2, TRKA, ULK1 and CLK4.

7. The method of claim 6, wherein said set of predictive kinases further comprises ANKK1.

8. The method of claim 7, wherein said set of predictive kinases further comprises JAK1.

9. The method of claim 1, wherein said test compound is tested at a concentration of about 10 μM.

10. The method of claim 1, wherein said set of predictive kinases further comprises AMPKA1 (BAA36547.1), CDK7 (NP_{—}001790.1), IKKE (NP_{—}054721.1), MLK2 (NP_{—}002437.2), MLK3 (NP_{—}002410.1), MERTK (AAB60430.1), MLCK (NP_{—}872299.1), PAK4 (NP_{—}001014833.1), SLK (NP_{—}055535.2), MST3 (NP_{—}003567.2), STK33 (NP_{—}112168.1), SYK (NP_{—}003168.2), TRKB (NP_{—}006171.2), TSSK1 (NP_{—}114417.1), and JAK2 (NP_{—}004963.1).

11. The method of claim 1, wherein inhibition of kinase activity of at least ten predictive kinases by 85% or greater constitutes a prediction that said compound would exhibit bone marrow toxicity in vivo.

12. The method of claim 11, wherein inhibition of kinase activity of at least fifteen predictive kinases by 85% or greater constitutes a prediction that said compound would exhibit bone marrow toxicity in vivo.

13. The method of claim 12, wherein inhibition of kinase activity of at least eighteen predictive kinases by 85% or greater constitutes a prediction that said compound would exhibit bone marrow toxicity in vivo.

14. The method of claim 13, wherein inhibition of kinase activity of nineteen predictive kinases by 85% or greater constitutes a prediction that said compound would exhibit bone marrow toxicity in vivo.

15. The method of claim 1, wherein inhibition of kinase activity is measured by determining the affinity of said compound for said predictive kinase.

16. A method for developing drugs, comprising:
a) providing a plurality of compounds;
b) determining the ability of each compound to inhibit the kinase activity of a set of predictive kinases, wherein each predictive kinase is selected from the group consisting of ANKK1, AURKC, CLK4, IRAK3, JAK1, MARK2, MUSK, MYLK2, RIPK1, ROCK2, STK17A, STK17B, SGK110, TRKA, TRKC, ULK1, ULK2, ZAP70, and TYK2; and
c) rejecting each compound that demonstrates inhibition of kinase activity of a threshold number of predictive kinases by about 85% or greater.

17. The method of claim 16, wherein said set of predictive kinases further comprises AMPKA1 (BAA36547.1), CDK7 (NP_{—}001790.1), IKKE (NP_{—}054721.1), MLK2 (NP_{—}002437.2), MLK3 (NP_{—}002410.1), MERTK (AAB60430.1), MLCK (NP_{—}872299.1), PAK4 (NP_{—}001014833.1), SLK (NP_{—}055535.2), MST3 (NP_{—}003567.2), STK33 (NP_{—}112168.1), SYK (NP_{—}003168.2), TRKB (NP_{—}006171.2), TSSK1 (NP_{—}114417.1), and JAK2 (NP_{—}004963.1).

18. The method of claim 17, wherein said threshold number of predictive kinases is fourteen.

19. The method of claim 18, wherein said threshold number of predictive kinases is sixteen.

20. The method of claim 19, wherein said threshold number of predictive kinases is eighteen.

21. The method of claim 16, wherein said threshold number of predictive kinases is nineteen.

22. The method of claim 16, wherein inhibition of kinase activity is measured by determining the affinity of said compound for said predictive kinase.
